(19) United States
(12) Plant Patent Application Publication
(10) Pub. No.: US 2002/0066128 P1
(43) Pub. Date: May 30, 2002

(54) Peach tree named "S-6368"

(76) Inventor: Rene Framcois Monet, Villenave D'Ornon (FR)

Correspondence Address: STRATTON BALLEW PLLC, 213 South 12th Avenue, Yakima, WA, 98902, US

(21) Appl. No.: 09/728,618

(22) Filed: Nov. 30, 2000

(51) Int. Cl. A01H 5/00

(52) U.S. Cl. PLT/197

(57) ABSTRACT

A new variety of peach tree is disclosed. The new variety is the result of a cross between Ferjalou Jalousia flat peach and Flavortop nectarine. The tree of the new variety is large and vigorous, with a spreading growth habit. The sweet, low acid fruit of the new variety is relatively large and has a broad oblate shape.

### DESCRIPTION OF THE VARIETY

The following is a detailed botanical description of the new and distinct variety of peach tree named “S-6368,” its flowers, foliage, and fruit as based on observations of specimens grown near Parker, Wash., United States of America. The new variety is the result of a controlled cross between Ferjalou Jalousia, flat peach and Flavortop nectarine. The new variety has been stably reproduced over successive generations. Discovery of the new variety and initial reproduction by in vitro propagation was carried out in a research nursery at Villenave d'Ornon, France. Subsequent asexual reproduction has taken place in Parker, Wash., U.S.A.

### ILLUSTRATION

The accompanying color photographic illustrations show typical specimens of the foliage and fruit of the new peach variety.

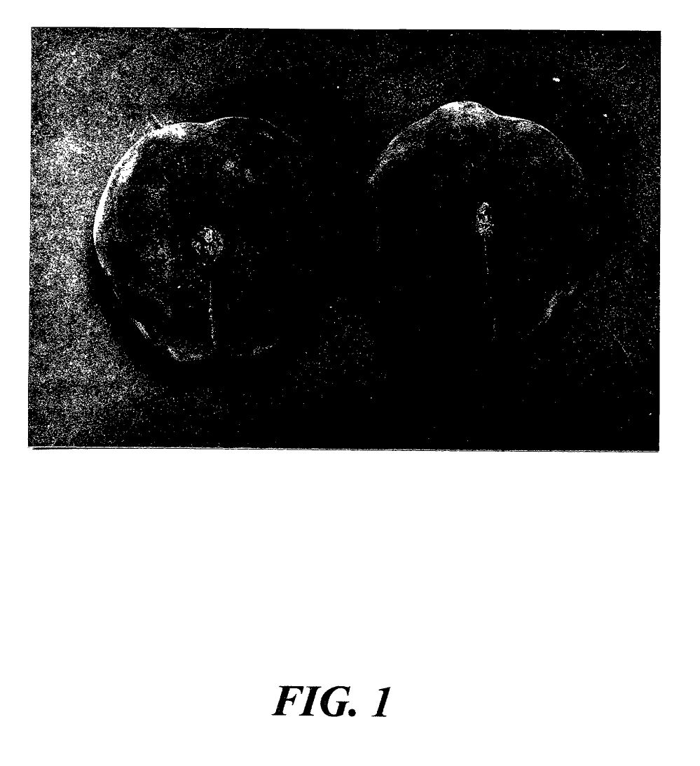

FIG. 1 shows representative fruit of the new peach tree;

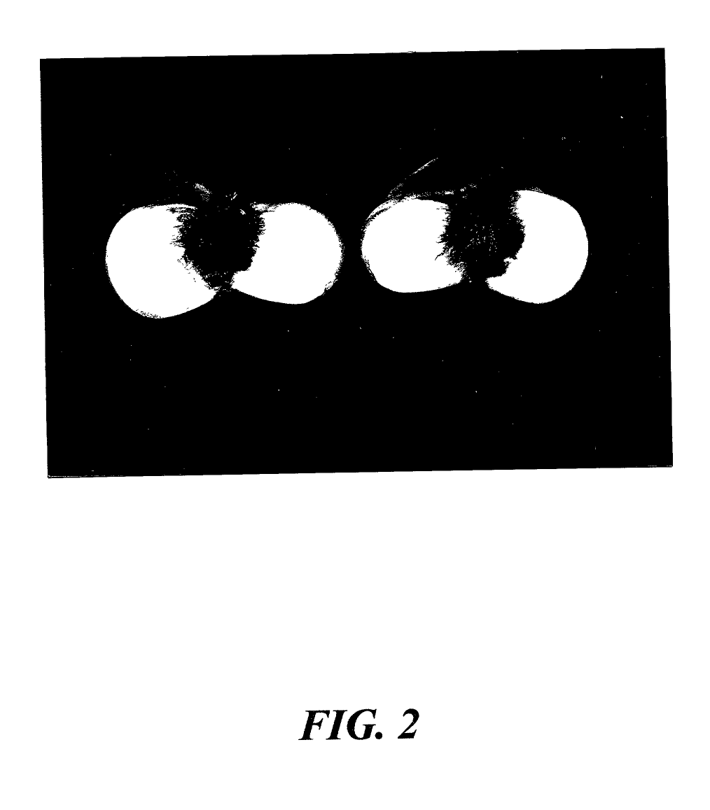

FIG. 2 shows the flesh of representative fruit of the new peach tree;

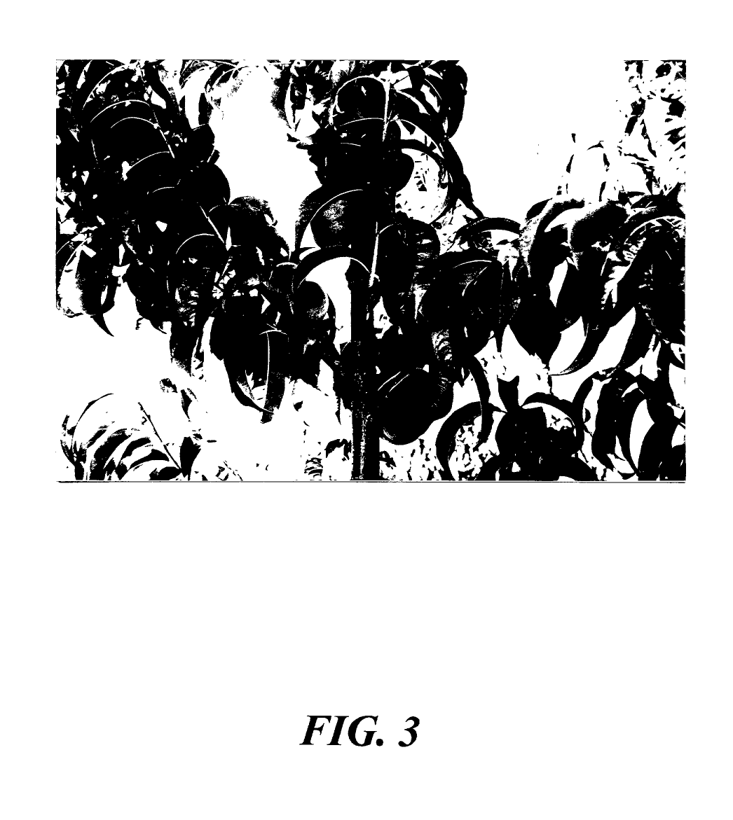

FIG. 3 shows a representative branch and trunk of the new peach tree;

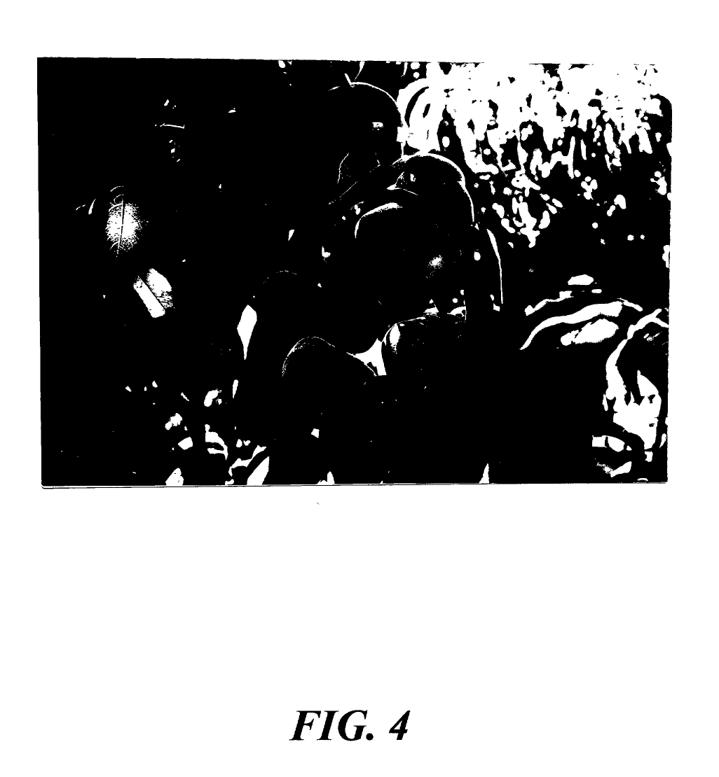

FIG. 4 shows representative fruit and foliage of the new peach tree;

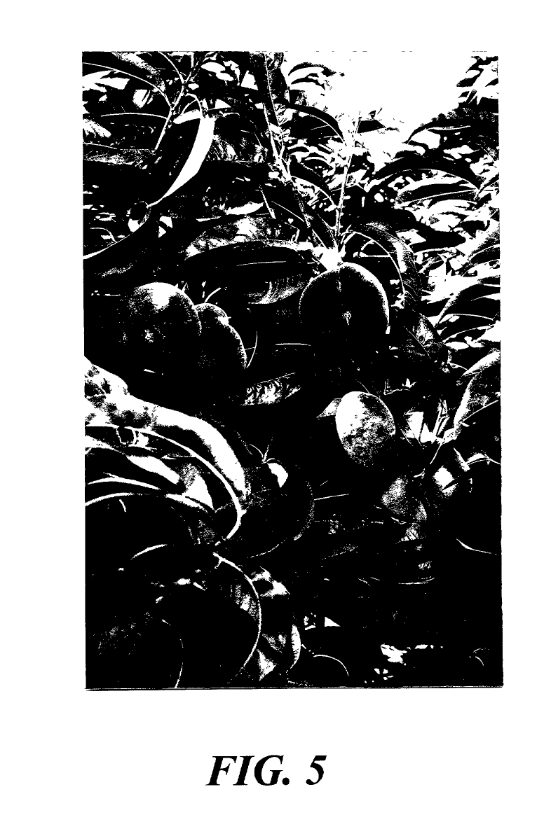

FIG. 5 shows representative fruit and foliage of the new peach tree; and

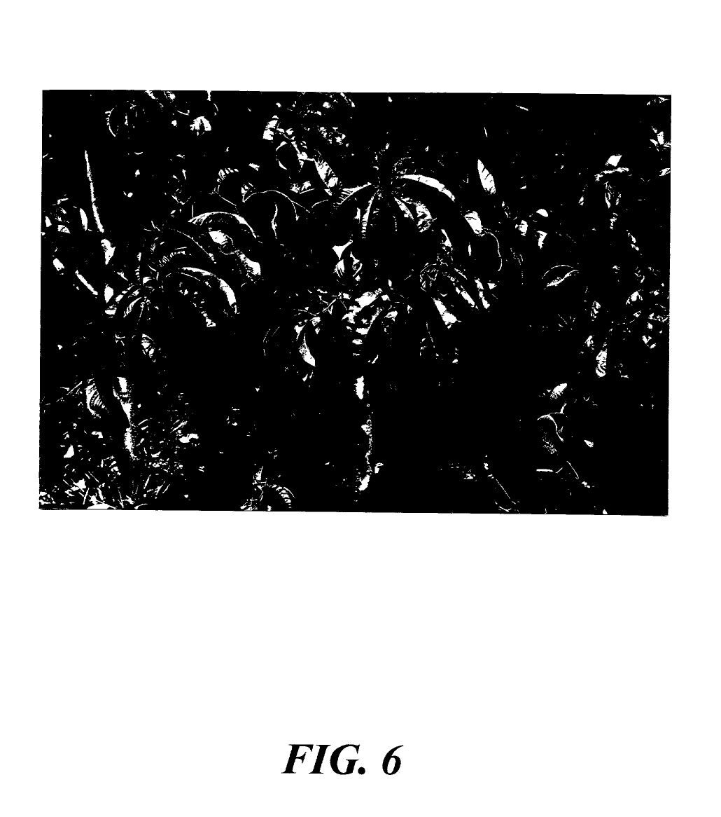

FIG. 6 shows representative fruit and foliage of the new peach tree.

### BOTANICAL DESCRIPTION

The following botanical description of the new variety is based on observations made in Parker, Wash., U.S.A. just prior to harvest (approximately September 10).

Tree:

Size.—Large.

Vigor.—Strong.

Habit.—Spreading.

Flowering shoot:

Thickness (excluding brindilles).—Medium.

Length of internodes.—Medium.

Anthocyanin coloration (side away from sun).—Weakly present.

Density of flower buds.—Medium.

General distribution of flower buds.—In groups of two or more.

Flower:

Type.—Showy.

Calyx Color of inner side (opened flower, before falling of petals).—Greenish yellow.

Corolla.—predominant color (inner side) Dark pink.

Petal shape.—Broad elliptic.

Petal size.—Large.

Number of petals.—Five.

Stamens position compared to petals.—Same level.

Stigma position compared to anthers.—Same level.

Anthers pollen.—Present.

Ovary pubescence.—Present.

Young shoot:

Length of stipule (fully expanded leaf).—Short.

Leaf blade:

Length.—Long.

Width.—Narrow.

Ratio.—Medium.

Shape in cross section.—Convex.

Recurvature of apex.—Present.

Angle at base.—Acute.

Angle at apex.—Medium.

Color.—Green.

Petiole:

Length.—Long.

Nectaries.—Present.

Shape of Nectaries.—Reniform.

Predominant number of nectaries.—Two.

Fruit:

Size.—Large (avg. mass 150g to 180g).

Shape (in ventral view).—Broad oblate.

Shape of pistil end.—Strongly depressed.

Symmetry (viewed from pistil end).—Asymmetric.

Prominence of suture.—Weak.

Depth of stalk cavity.—Shallow.

Width of stalk cavity.—Broad.

Ground color.—Cream yellow.

Over color.—Present.

Hue of over color.—Medium red.

Pattern of over color.—Solid flush.

Extent of over color.—Large.

Pubescence.—Present.

Density of pubescence.—Medium.

Thickness of skin.—Medium.

Adherence of skin to flesh.—Weak.

Firmness of flesh.—Medium.

Ground color of flesh.—Orange.

Anthocyanin coloration directly under skin.—Absent or very weakly expressed.

Anthocyanin coloration of flesh.—Absent or very weakly expressed.

Anthocyanin coloration around stone.—Weakly expressed.

Texture of the flesh.—Not fibrous.

Sweetness.—High.

Acidity.—Low.

Stone:

Size compared to fruit.—Small.

Shape (in lateral view).—Oblate.

Intensity of brown color.—Light.

Relief of surface.—Pits and grooves.

Tendency of splitting (at peak harvest).—Absent or very low.

Adherence to flesh.—Present.

Degree of adherence to flesh.—Strong.

Time of leaf bud burst.—Medium.

Time of beginning of flowering.—Medium.

Duration of flowering.—Medium.

Time of maturity for consumption.—Medium.

Tendency to preharvest drop.—Weak.

Resistance to pests and disease.—Unknown.

Virus status.—Unknown.

Propagation method.—In-vitro.

Although this new variety of peach tree possesses the described characteristics under the ecological conditions which exist in the area of Parker, Wash., United States of America, where it was grown and tested, it is to be expected that variations of these characteristics may occur when grown in areas with different climatic conditions, soil types and/or varying cultural practices.

What is claimed is:

1. A new and distinct variety of peach tree, substantially as illustrated and described herein.
